(19) United States
(12) Patent Application Publication
(10) Pub. No.: US 2013/0118570 A1
(43) Pub. Date: May 16, 2013

(54) DYE FOR PHOTOELECTRIC CONVERSION, SEMICONDUCTOR ELECTRODE, PHOTOELECTRIC CONVERSION ELEMENT, SOLAR CELL, AND NOVEL PYRROLINE-BASED COMPOUND

(71) Applicants: Katsumi Maeda, Tokyo (JP); Shin Nakamura, Tokyo (JP); Kentaro Nakahara, Tokyo (JP)

(72) Inventors: Katsumi Maeda, Tokyo (JP); Shin Nakamura, Tokyo (JP); Kentaro Nakahara, Tokyo (JP)

(73) Assignee: NEC CORPORATION, Tokyo (JP)

(21) Appl. No.: 13/520,928

(22) PCT Filed: Nov. 18, 2010
(86) PCT No.: PCT/JP2010/006773

§ 371 Date: Jul. 6, 2012

(30) Foreign Application Priority Data
Jan. 7, 2010 (JP) 2010-002241

(51) Int. Cl. C07D 207/44; H01L 51/42; C07D 403/04; H01L 51/44

(52) U.S. Cl. CPC C07D 207/44; H01L 51/441; H01L 51/42; C07D 403/04
USPC 136/256; 136/263; 548/550; 548/466

(57) ABSTRACT

Provided is a dye for photoelectric conversion containing at least one or more kind of a compound represented by the following General Formula (1) (in Formula (1), R¹ and R² represent any one of —CN, —SO₂R, —COOR, and —CONR₂ (R represents a hydrogen atom, a substituted or unsubstituted alkyl group, a cycloalkyl group, or an aryl group); R³ represents a direct bond or a substituted or unsubstituted alkylene group; X represents an acidic group; and D represents an organic group having an electron donating substituent or a substituted or unsubstituted heterocyclic group).

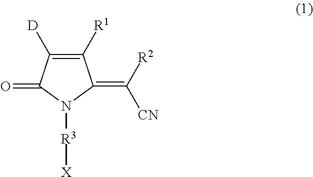

### TECHNICAL FIELD

The present invention relates to a dye for photoelectric conversion, a semiconductor electrode, a photoelectric conversion element, a solar cell, and a novel pyrroline-based compound.

### BACKGROUND ART

Since a large amount of fossil fuels represented by petroleum have been used so far, the level of CO₂ has increased. Consequently, global warming has become a serious problem, and there is a concern over the depletion of fossil fuels. Accordingly, how the demand for a large amount of energy can be met in the future has become an important global issue. In this situation, for power generation, the use of sunlight which is infinite and does not produce hazardous substances as nuclear power generation does is being positively examined. As solar cells that convert light energy into electric energy, inorganic solar cells such as monocrystalline silicon, polycrystalline silicon, and amorphous silicon as well as organic solar cells using organic dyes and conductive polymer materials have been proposed.

In this situation, a dye-sensitized solar cell (Grätzel solar cell) proposed in 1991 by Dr. Grätzel et al. from Switzerland (Non-patent Document 1 and Patent Document 1) is produced in a simple process and yields conversion efficiency as good as amorphous silicon. Therefore, the dye-sensitized solar cell is expected to be a solar cell for the next generation. The Grätzel's dye-sensitized solar cell includes a semiconductor electrode that is prepared by forming a semiconductor layer onto which a dye has been adsorbed on a conductive substrate, a counter electrode that faces this electrode and is formed of a conductive substrate, and an electrolyte layer that is held between both the electrodes.

In this electric cell, the adsorbed dye is excited by absorbed light, and electrons are injected to the semiconductor layer from the excited dye. The dye is oxidized when the electrons are released and returns to the original dye when the electrons move to the dye by the oxidization reaction of a redox agent in the electrolyte layer. The redox agent donating electrons to the dye is reduced again in the counter electrode. Due to this series of reactions, the dye-sensitized solar cell functions as an electric cell.

In the Grätzel's dye-sensitized solar cell, porous titanium oxide obtained by sintering fine particles is used in the semiconductor layer. Consequently, the solar cell has characteristics that an effective reactive surface area is increased by about 1000 times and that larger photocurrents are obtained compared to the related art. In the Grätzel's dye-sensitized solar cell, a ruthenium complex is used as a sensitizing dye, and specifically, a cis-bis(isothiocyanato)-bis-(2,2′-bipyridyl-4,4′-dicarboxylic acid) ruthenium(II) bis(tetrabutylammonium) complex, a bipyridine complex of ruthenium such as cis-bis(isothiocyanato)-bis-(2,2′-bipyridyl-4,4′-dicarboxylic acid)ruthenium(II), and a tris(isothiocyanato) (2,2′:6′,2″-terpyridyl-4,4′,4″-tricarboxylic acid)ruthenium(II) tristetrabutylammonium complex which is a type of terpyridine complex are used.

In addition, Patent Document 6 discloses a novel merocyanine dye and a method of producing the same.

### RELATED DOCUMENT

### Patent Document

[Patent Document 1] Japanese Patent No. 2664194
[Patent Document 2] JP-A-2004-95450
[Patent Document 3] JP-A-2001-76773
[Patent Document 4] JP-A-11-238905
[Patent Document 5] JP-A-2005-19252
[Patent Document 6] JP-A-9-255883

### Non-Patent Document

[Non-patent Document 1] Brian O'Regan, Michael Grätzel, “A low-cost, high-efficiency solar cell based on dye-sensitized colloidal TiO₂ films”, Nature, United Kingdom, Nature Publishing Group, Oct. 24, 1991, Vol. 353, pp 737-740

### DISCLOSURE OF THE INVENTION

The dye-sensitized solar cell using a ruthenium complex has a problem in that a precious metal ruthenium is used for the raw material of the dye. When the dye-sensitized solar cell is produced in a large quantity by using the ruthenium complex, restriction on resources becomes a problem, and the solar cell becomes expensive, whereby the solar cells cannot come into widespread use.

Therefore, in recent years, a large number of organic dyes of a non-ruthenium complex have been proposed as the sensitizing dyes in the dye-sensitized solar cell. Examples of such organic dyes include a coumarin-based dye (Patent Document 2), a cyanine-based dye (Patent Document 3), a merocyanine-based dye (Patent Documents 4 and 5), and the like. Compared to a ruthenium complex, these organic dyes have a larger molar absorbance coefficient, and molecules of these organic dyes can be more freely designed. Accordingly, these dyes have raised expectation of development of dyes having a high photoelectric conversion efficiency. However, these organic dyes have a problem in that high photoelectric conversion efficiency is not easily obtained compared to a ruthenium complex.

The present invention has been made to solve the above problem, and an object thereof is to provide a pyrroline-based compound with excellent photoelectric conversion characteristics, a dye for photoelectric conversion, a semiconductor electrode, a photoelectric conversion element, and a solar cell.

According to the present invention, there is provided a dye for photoelectric conversion containing at least one or more kind of a compound represented by the following General Formula (1).

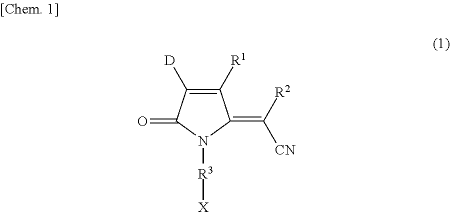

(in Formula (1), R¹ and R² represent any one of —CN, —SO₂R, —COOR, and —CONR₂ (R represents a hydrogen atom, a substituted or unsubstituted alkyl group, a cycloalkyl group, or an aryl group); R³ represents a direct bond or a substituted or unsubstituted alkylene group; X represents an acidic group; and D represents an organic group having an electron donating substituent or a substituted or unsubstituted heterocyclic group)

According to the present invention, there is provided a semiconductor electrode having a semiconductor layer onto which at least one or more kind of the dye for photoelectric conversion has been adsorbed.

According to the present invention, there is provided a photoelectric conversion element using the semiconductor electrode.

According to the present invention, there is provided a solar cell including the photoelectric conversion element.

According to the present invention, there is provided a compound represented by the following General Formula (1).

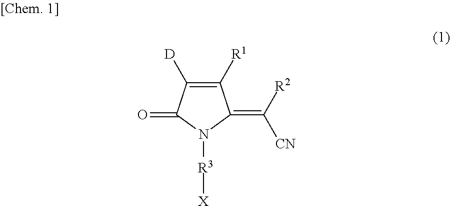

(in Formula (1), R¹ and R² represent any one of —CN, —SO₂R, —COOR, and —CONR₂ (R represents a hydrogen atom, a substituted or unsubstituted alkyl group, a cycloalkyl group, or an aryl group); R³ represents a direct bond or a substituted or unsubstituted alkylene group; X represents an acidic group; and D represents an aryl group having an electron donating substituent or a substituted or unsubstituted heterocyclic group)

According to the present invention, a pyrroline-based compound with excellent photoelectric conversion characteristics, a dye for photoelectric conversion, a semiconductor electrode, a photoelectric conversion element, and a solar cell are realized.

### BRIEF DESCRIPTION OF THE DRAWINGS

FIG. 1 is a cross-sectional view schematically illustrating an exemplary constitution of the photoelectric conversion element of the present embodiment.

### DESCRIPTION OF EMBODIMENTS

Hereinbelow, the embodiments of the present invention will be described in detail.

<Pyrroline-Based Compound>

The compound of the present embodiment is a pyrroline-based compound represented by the following General Formula (1).

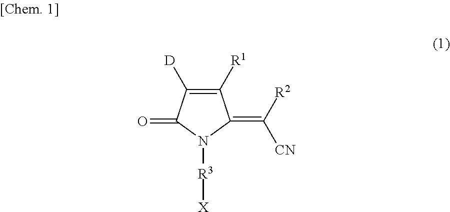

In Formula (1), R¹ and R² represent any one of —CN, —SO₂R, —COOR, and —CONR₂. R represents a hydrogen atom, a substituted or unsubstituted alkyl group (for example, a methyl group, an ethyl group, a propyl group, a butyl group, or the like), a cycloalkyl group (for example, a cyclopentyl group, a cyclohexyl group, or the like), or an aryl group (for example, a phenyl group, a tolyl group, a naphthyl group, or the like). R³ represents a direct bond or a substituted or unsubstituted alkylene group (for example, a methylene group, an ethylene group, a propylene group, a butylene group, or the like, and among these, an alkylene group having not more than 2 carbon atoms is preferable).

X represents an acidic group (for example, a carboxy group, a hydroxy group, a sulfonic acid group, a phosphonic acid, or the like, and among these, a carboxy group is preferable). When a semiconductor electrode is produced, the pyrroline-based compound represented by General Formula (1) is used by being adsorbed onto a semiconductor layer. Accordingly, this compound needs to have in a molecule a functional group that can be adsorbed onto the semiconductor layer. In the pyrroline-based compound of the present embodiment, the acidic group represented by X plays a role of the functional group.

D represents an organic group having an electron donating substituent or a substituted or unsubstituted heterocyclic group. Examples of the organic group having an electron donating substituent include an electron donating substituent and a group obtained when the electron donating substituent is substituted with an organic group other than an electron donating group. Examples of the organic group other than an electron donating group include an aryl group.

The aryl group in D is a monovalent aromatic hydrocarbon group. Examples of the aromatic ring include aromatic rings having 6 to 22 carbon atoms, such as benzene, naphthalene, anthracene, indene, azulene, fluorene, and phenanthrene. These aryl groups may further have a substituent other than the electron donating substituent.

Examples of the heterocycle of the heterocyclic group in D include indole, carbazole, furan, thiophene, pyrrole, pyridine, quinoline, imidazole, oxazole, isoxazole, thiazole, isothiazole, pyrazole, acridine, phenoxazine, xanthene, benzoxazole, benzothiazole, benzimidazole, and the like. These heterocyclic groups may further have a substituent.

Examples of the electron donating substituent in D include an amino group which may have a substituent, a hydroxy group, an alkoxy group, and the like. The amino group which may have a substituent is preferably a disubstituted amino group. In a case of the disubstituted amino group, the substituents may form a ring.

Specific examples of Dare shown in the following Tables 1-1 and 1-2.

TABLE 1-1

| | |
|---|---|
| D1 | 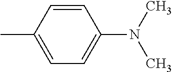 |
| D2 | 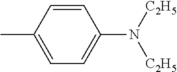 |
| D3 | 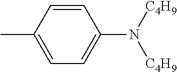 |
| D4 | 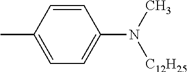 |
| D5 | 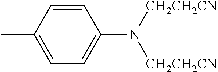 |
| D6 | 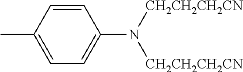 |
| D7 | 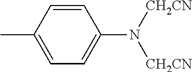 |
| D8 | 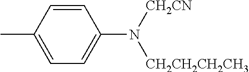 |
| D9 | 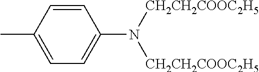 |
| D10 | 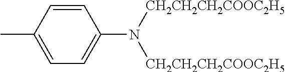 |
| D11 | 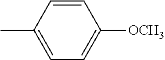 |
| D12 | 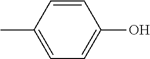 |

TABLE 1-2

| | |
|---|---|
| D13 | 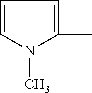 |
| D14 | 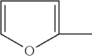 |
| D15 | 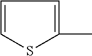 |
| D16 | 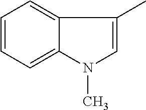 |
| D17 | 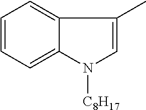 |
| D18 | 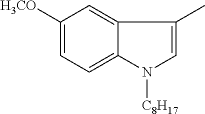 |
| D19 | 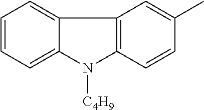 |
| D20 | 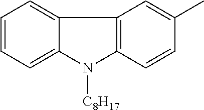 |
| D21 | 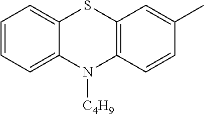 |
| D22 | 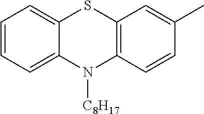 |
| D23 | 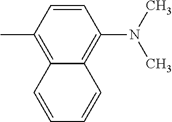 |
| D24 | 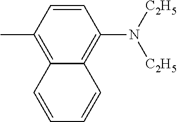 [SMILES: CCN(CC)c1ccc(C)c2ccccc12] |

Examples of the structure of the pyrroline-based compound represented by General Formula (1) other than D are shown in Table 2.

TABLE 2

| | |
|---|---|
| A1 | 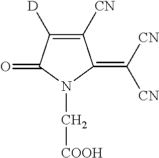 [SMILES: [2H]C1=C(C#N)C(=C(C#N)C#N)N(CC(=O)O)C1=O] |
| A2 | 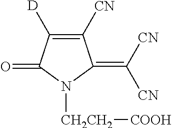 |
| A3 | 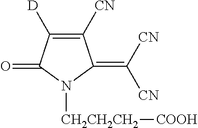 |
| A4 | 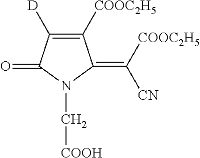 |
| A5 | 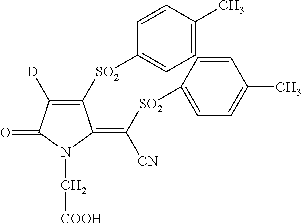 |
| A6 | 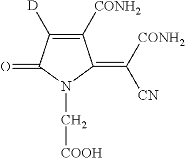 [SMILES: [2H]C1=C(C(N)=O)/C(=C(/C#N)C(N)=O)N(CC(=O)O)C1=O] |
| A7 | 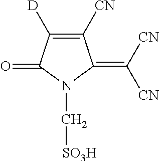 [SMILES: [2H]C1=C(C#N)C(=C(C#N)C#N)N(CS(=O)(=O)O)C1=O] |
| A8 | 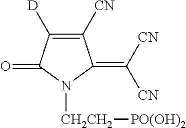 |

<Photoelectric Conversion Element>

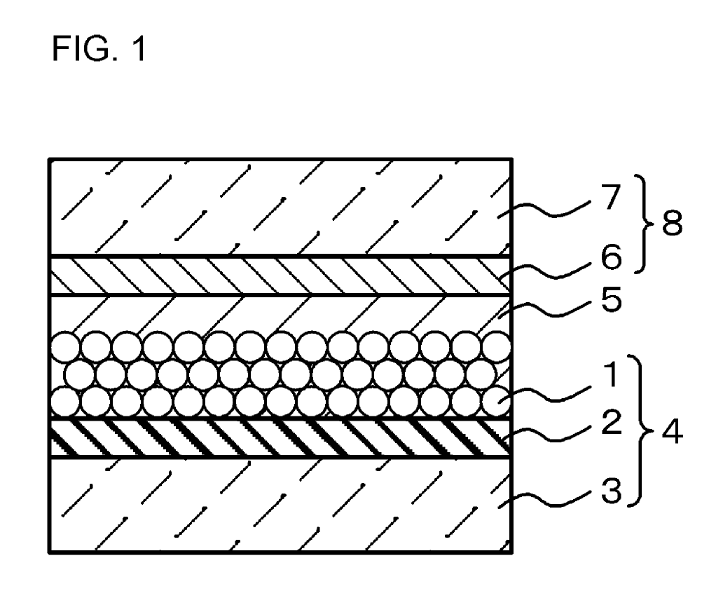

FIG. 1 shows a cross-sectional view that schematically illustrates an exemplary constitution of the photoelectric conversion element of the present embodiment. The photoelectric conversion element shown in FIG. 1 includes a semiconductor electrode 4, a counter electrode 8, and an electrolyte layer 5 held between both the electrodes. The semiconductor electrode 4 includes a light transmissive substrate 3, a transparent conductive layer 2, and a semiconductor layer 1. The counter electrode 8 includes a catalytic layer 6 and a substrate 7. In addition, a dye has been adsorbed onto the semiconductor layer 1.

When light enters the photoelectric conversion element of the present embodiment, the dye adsorbed onto the semiconductor layer 1 is excited, whereby electrons are released. The electrons move to a conduction band of the semiconductor and then further move to the transparent conductive layer 2 by diffusion. The electrons in the transparent conductive layer 2 move to the counter electrode 8 via an external circuit (not shown in the drawing) and. The electrons then passes through the electrolyte layer 5 and return to the oxidized dye, whereby the dye is regenerated. The photoelectric conversion element is constituted in this manner to function as an electric cell. The respective constituent elements will be described below based on FIG. 1 for example.

<Semiconductor Electrode>

The semiconductor electrode 4 includes a light transmissive substrate 3, the transparent conductive layer 2, and the semiconductor layer 1. FIG. 1 illustrates a constitution in which the light transmissive substrate 3, the transparent conductive layer 2, and the semiconductor layer 1 are laminated in this order toward the inside of the element from the outside of the element. Moreover, a dye (not shown in FIG. 1) is adsorbed onto the semiconductor layer 1.

<Conductive Substrate>

The conductive substrate may have either a single layer structure in which the substrate itself has conductivity or a double layer structure in which a conductive layer is formed on the substrate. FIG. 1 illustrates an example of a conductive substrate having a double layer structure in which the transparent conductive layer 2 is formed on the light transmissive substrate 3. Examples of the substrate include a glass substrate, a plastic substrate, a metal plate, and the like. Among these, a substrate having high light transmittance, for example, a transparent substrate is particularly preferable. Examples of materials of the transparent plastic substrate include polyethylene terephthalate (PET), polyethylene naphthalate (PEN), polycarbonate (PC), polycycloolefin, polyphenylene sulfide, and the like. The type of the conductive layer formed on the substrate (for example, the light transmissive substrate 3) is not particularly limited, but for example, the transparent conductive layer 2 constituted with a transparent material such as Indium-Tin-Oxide (ITO), Florine doped Tin Oxide (FTO), Indium Zinc Oxide (IZO), or tin oxide (SnO₂) is preferable. The transparent conductive layer 2 may be formed in a film shape on the entire surface or a portion of the surface of the substrate. The film thickness or the like of the transparent conductive layer 2 can be appropriately selected, but the film thickness is preferably about equal to or more than 0.02 μm and equal to or less than 10 μm. Since the method of preparing this transparent conductive layer 2 can be realized by using known techniques, the description will not be repeated.

For the purpose of decreasing resistance of the conductive substrate, the conductive substrate of the present embodiment can use a metal lead wire. Examples of materials of the metal lead wire include metals such as aluminum, copper, gold, silver, platinum, and nickel. The metal lead wire may be prepared in a method in which the metal lead wire is prepared by vapor-deposition, sputtering, or the like and ITO or FTO is provided on the wire. Alternatively, the metal lead wire may be prepared on the transparent conductive layer after the transparent conductive layer 2 is provided on the substrate (for example, the light transmissive substrate 3).

Hereinbelow, description will be made based on conductive substrates 2 and 3 having a double layer structure in which the transparent conductive layer 2 is formed on the light transmissive substrate 3.

<Semiconductor Layer>

As materials constituting the semiconductor layer 1, a single semiconductor such as silicon or germanium, metal chalcogenide, a compound having a perovskite structure, and the like can be used. Examples of metal chalcogenides include oxides of titanium, tin, zinc, iron, tungsten, indium, zirconium, vanadium, niobium, tantalum, strontium, hafnium, cerium, or lanthanum; sulfides of cadmium, zinc, lead, silver, antimony, or bismuth; selenides of cadmium or lead, telluride of cadmium, and the like. Examples of other compound semiconductors include phosphides of zinc, gallium, indium, cadmium, and the like; gallium arsenide; copper-indium-selenide; copper-indium-sulfide; and the like. Examples of compounds having a perovskite structure include known semiconductor materials such as barium titanate, strontium titanate, and potassium niobate. These semiconductor materials may be used alone or used as a mixture of 2 or more kinds thereof. Among these, the semiconductor layer 1 is preferably constituted with a semiconductor material containing titanium oxide or zinc oxide, and most preferably constituted with a semiconductor material containing titanium oxide, from the viewpoints of conversion efficiency, stability, and safety. More specific examples of titanium oxide include various titanium oxides such as anatase type titanium oxide, rutile type titanium oxide, amorphous titanium oxide, metatitanic acid, and orthotitanic acid; titanium oxide-containing complexes; and the like. Among these, anatase type titanium oxide is preferable from the viewpoint of further improving the stability of photoelectric conversion.

Examples of the shape of the semiconductor layer 1 include a porous semiconductor layer that is obtained by sintering fine semiconductor particles or the like, and a thin film-like semiconductor layer that is obtained by a sol-gel method, a sputtering method, a spray thermal decomposition method, or the like. In addition, the semiconductor layer 1 may be formed of another fiber-like semiconductor layer or needle-like crystals. The shape of the semiconductor layer 1 can be appropriately selected according to the usage purpose of the photoelectric conversion element. Among the above semiconductors, the semiconductor layer 1 having a large specific surface area, such as the porous semiconductor layer or the semiconductor layer formed of needle-like crystals is preferable from the viewpoint of the amount of a dye adsorbed or the like. Moreover, from the viewpoint that the rate of utilization of incident light can be adjusted by the particle size of the fine semiconductor particles, it is preferable to use the porous semiconductor layer formed of fine semiconductor particles as the semiconductor layer 1. The semiconductor layer 1 may be single layered or multi layered. If the semiconductor layer 1 is made multi layered, it is possible to more easily form the semiconductor layer 1 having a sufficient thickness. The multi layered porous semiconductor layer 1 formed of fine semiconductor particles may be formed of a plurality of semiconductor layers differing in the average particle size of the fine semiconductor particles. For example, the average particle size of fine semiconductor particles of a semiconductor layer (a first semiconductor layer) close to a light incident side may be made smaller than that of a semiconductor layer (a second semiconductor layer) far from the light incident side. In this case, a large amount of light is absorbed into the first semiconductor layer, the light passing through the first semiconductor layer is efficiently scattered in the second semiconductor layer so as to be returned to the first semiconductor layer, and the returned light is absorbed into the first semiconductor, whereby it is possible to further improve the entire light absorptance. Though not particularly limited, the film thickness of the semiconductor layer 1 is set to, for example, equal to or more than 0.5 μm and equal to or less than 45 μm, from the viewpoints of transmittance, conversion efficiency, and the like. The specific surface area of the semiconductor layer 1 can be set to, for example, equal to or more than 10 m²/g and equal to or less than 200 m²/g, from the viewpoint of causing a large amount of dye to be adsorbed.

In the constitution in which a dye has been adsorbed onto the porous semiconductor layer 1, in order to transport charge by causing ions in the electrolyte to be more sufficiently diffused, the porosity of the porous semiconductor layer 1 is preferably set to, for example, equal to or more than 40% and equal to or less than 80%. The porosity refers to a volumetric proportion of the pores in the semiconductor layer 1 to the volume of the semiconductor layer 1, which is expressed in terms of a percentage.

<Method of Forming Semiconductor Layer>

Next, a method of forming the semiconductor layer 1 will be described based on the porous semiconductor layer 1 for example. For the porous semiconductor layer 1, for example, fine semiconductor particles are added to a dispersion medium such as an organic solvent or water together with an organic compound such as a resin and a dispersant so as to prepare a suspension. This suspension is then coated onto a conductive substrate (transparent conductive layer 2 in FIG. 1), followed by drying and baking, thereby forming the semiconductor layer 1. If an organic compound is added to a dispersion medium in advance together with fine semiconductor particles, the organic compound is combusted during baking, whereby more sufficient gaps can be secured inside the porous semiconductor layer 1. In addition, it is possible to vary the porosity by controlling the molecular weight of the organic compound combusted during baking or the amount of the organic compound added.

Any compound can be used as the organic compound to be used as long as the compound is dissolved in the suspension and can be removed by being combusted during baking. Examples of the organic compound include polymers or copolymers of vinyl compounds such as polyethylene glycol, a cellulose ester resin, a cellulose ether resin, an epoxy resin, a urethane resin, a phenol resin, a polycarbonate resin, a polyarylate resin, a polyvinyl butyral resin, a polyester resin, a polyvinyl formal resin, a silicon resin, styrene, vinyl acetate, acrylic acid ester, and methacrylic acid ester. The type and amount of the resin can be appropriately selected and adjusted according to the condition of the fine particles to be used, the total weight of the entire suspension, and the like. Here, when the proportion of the fine semiconductor particles is 10 wt % or more based on the total weight of the entire suspension, the strength of the prepared film can be more sufficiently enhanced. In addition, when the proportion of the fine semiconductor particles is 40 wt % or less based on the total weight of the entire suspension, the porous semiconductor layer 1 having a high porosity can be more stably obtained. Consequently, the proportion of the fine semiconductor particles is preferably set to equal to or more than 10 wt % and equal to or less than 40 wt %, based on the total weight of the entire suspension.

As the fine semiconductor particles, it is possible to use particles of a single compound semiconductor or a plurality of compound semiconductors having appropriate average particle size, for example, an average particle size of about equal to or more than 1 nm and equal to or less than 500 nm, and the like. Among these, particles having an average particle size of about equal to or more than 1 nm and equal to or less than 50 nm are desirable, in respect of increasing the specific surface area. Moreover, in order to increase the rate of utilization of incident light, semiconductor particles having a relatively large average particle size of about equal to or more than 200 nm and equal to or less than 400 nm may be added.

Examples of the method of producing fine semiconductor particles include a sol-gel method such as a hydrothermal synthesis method, a sulfuric acid method, a chlorine method, and the like. Any method can be used as long as the method can produce target fine particles, but it is preferable to synthesize the particles by a hydrothermal synthesis method from the viewpoint of crystallinity.

Examples of dispersion media of the suspension include glyme-based solvents such as ethylene glycol monomethyl ether; alcohols such as isopropyl alcohol; mixed solvents such as isopropyl alcohol/toluene; water; and the like.

Examples of methods of coating the suspension include known methods such as a doctor blade method, a squeegee method, a spin coater method, and a screen printing method. After the suspension is coated, the coating film is dried and baked. The condition of the drying and baking is set such that the drying and baking are performed in the atmosphere or in an inert gas atmosphere within a temperature range of about equal to or higher than 50° C. and equal to or lower than 800° C. for about equal to or longer than 10 seconds and equal to or shorter than 12 hours. The drying and baking can be performed once at a constant temperature or performed twice by changing temperature.

So far, the method of forming the porous semiconductor layer 1 has been described in detail, but another type of semiconductor layer 1 can also be formed by using various known methods.

<Dye>

The dye in the photoelectric conversion element of the present embodiment uses the above-described pyrroline-based compound of the present embodiment represented by General Formula (1).

Examples of methods of causing the dye to be adsorbed onto the semiconductor layer 1 include a method of dipping a semiconductor substrate, that is, the conductive substrates 2 and 3 including the semiconductor layer 1 into a solution in which the dye is dissolved, and a method of causing the dye to be adsorbed by coating the dye solution onto the semiconductor layer 1.

Examples of solvents of the solution include nitrile-based solvents such as acetonitrile, propionitrile, and methoxyacetonitrile; alcohol-based solvents such as methanol, ethanol, and isopropyl alcohol; ketone-based solvents such as acetone, methyl ethyl ketone, methyl isobutyl ketone, and cyclohexanone; ester-based solvents such as ethyl acetate and butyl acetate; ether-based solvents such as tetrahydrofuran and dioxane; amide-based solvents such as N,N-dimethylformamide, N,N-dimethylacetamide, and N-methyl-2-pyrrolidone; halogen-based solvents such as dichloromethane, chloroform, dichloroethane, trichloroethane, and chlorobenzene; hydrocarbon-based solvents such as toluene, xylene, cyclohexane; water; and the like. These solvents may be used alone or used as a mixture of 2 or more kinds thereof.

When the semiconductor substrate is dipped into the dye solution for a certain time, the solution can be stirred or refluxed under heating, or ultrasonic waves can be applied to the solution. In addition, after the dye is adsorbed, it is desirable to wash the substrate with a solvent such as alcohol so as to remove the dye remaining without being adsorbed.

The amount of the dye supported is preferably within a range of equal to or more than 1×10⁻¹⁰ mol/cm² and equal to or less than 1×10⁻⁴ mol/cm², and particularly preferably within a range of equal to or more than 1×10⁻⁹ mol/cm² and equal to or less than 9.0×10⁻⁶ mol/cm². This is because an effect of improving photoelectric conversion efficiency can be obtained sufficiently and economically within this range.

In order to widen the wavelength band as much as possible in which photoelectric conversion can be performed and to improve the conversion efficiency, two or more kinds of dyes may be used as a mixture. In this case, it is preferable to appropriately select the types and proportions of the dyes, in consideration of the absorption wavelength band and strength of the dye.

Moreover, in order to inhibit the decrease in conversion efficiency caused by mingling of dyes, additives may be concurrently used when the dye is adsorbed. Examples of the additives include steroid-based compounds (for example, deoxycholic acid, cholic acid, chenodeoxycholic acid, and the like) having a carboxy group.

<Counter Electrode>

In the present embodiment, the counter electrode 8 includes the catalytic layer 6 on the substrate 7. In the photoelectric conversion element of the present embodiment, holes that are generated from the dye adsorbed onto the semiconductor layer 1 due to the incidence of light are transported to the counter electrode 8 through the electrolyte layer 5. However, the material of the counter electrode 8 is not limited as long as the counter electrode 8 carries out a function by which electrons and holes effectively annihilate each other. As the catalytic layer 6 of the counter electrode 8, a metal vapor deposition film formed on the substrate 7 by a vapor deposition method or the like can be used. For example, the catalytic layer 6 may be a Pt layer formed on the substrate 7. In addition, the catalytic layer 6 of the counter electrode 8 may contain a nanocarbon material. For example, a paste containing carbon nanotubes, carbon nanohorns, or carbon fibers may be sintered on a porous insulating film so as to form the catalytic layer 6 of the counter electrode 8. The nanocarbon material has a large specific surface area and can improve the probability of annihilation between electrons and holes. Examples of the substrate 7 include transparent substrates such as glass and a polymer film, metal plates (foils), and the like. The light transmissive counter electrode 8 can be prepared by selecting transparent conductive film-attached glass as the substrate 7 and forming the catalytic layer 6 of platinum or carbon on the substrate by using a vapor deposition method or a sputtering method.

<Electrolyte Layer>

The electrolyte layer 5 used in the present embodiment needs to have a function of transporting holes that are generated from the dye adsorbed onto the semiconductor layer 1 due to the incidence of light to the counter electrode 8. For the electrolyte layer 5, it is possible to use an electrolytic solution prepared by dissolving a redox pair in an organic solvent, a gel electrolyte prepared by impregnating a polymer matrix with a liquid that is obtained by dissolving a redox pair in an organic solvent, a molten salt containing a redox pair, a solid electrolyte, an organic hole-transporting material, and the like. In addition, the electrolyte layer 5 can be constituted with an electrolyte, a solvent, and additives.

Examples of the electrolyte include metallic iodides such as LiI, NaI, KI, CsI, and CaI₂; a combination of iodides such as iodine salts of quaternary ammonium compounds, such as tetraalkylammonium iodide, pyridinium iodide, imidazolium iodide, and the like with I₂; metallic bromides such as LiBr, NaBr, KBr, CsBr, and CaBr₂; a combination of bromides such as bromine salts of quaternary ammonium compounds, such as tetraalkylammonium bromide, pyridinium bromide, and the like with Br₂; metal complexes such as ferrocyanic acid salt-ferricyanic acid salt and ferrocene-ferrocenium ion; sulfur compounds such as sodium polysulfide and alkyl thiol-alkyl disulfide; viologen dyes; hydroquinone-quinone; and the like. Among these, a combination of LiI, pyridinium iodide, or imidazolium iodide with I₂ is preferable. These electrolytes may be used alone or used as a mixture of 2 or more kinds thereof. It is also possible to use a molten salt that stays in a molten state at ambient temperature as an electrolyte, and in this case, a solvent may not be particularly used.

Examples of solvents of the electrolyte layer 5 include carbonate-based solvents such as ethylene carbonate, diethyl carbonate, dimethyl carbonate, and propylene carbonate; amide-based solvents such as N-methyl-2-pyrrolidone and N,N-dimethylformamide; nitrile-based solvents such as methoxypropionitrile, propionitrile, methoxyacetonitrile, and acetonitrile; lactone-based solvents such as γ-butyrolactone and valerolactone; ether-based solvents such as tetrahydrofuran, dioxane, diethyl ether, and ethylene glycol dialkyl ether; alcohol-based solvents such as methanol, ethanol, and isopropyl alcohol; non-protonic polar solvents such as dimethyl sulfoxide and sulfolane; heterocyclic compounds such as 2-methyl-3-oxazolidinone and 2-methyl-1,3-dioxolane; and the like. These solvents may be optionally used as a mixture of 2 or more kinds thereof.

In the present embodiment, basic additives may be added to the electrolyte layer 5 so as to inhibit dark currents. The type of the basic additive is not particularly limited, and examples thereof include t-butylpyridine, 2-picoline, 2,6-lutidine, and the like. When a basic compound is added, the concentration of this compound added is set to about, for example, equal to or more than 0.05 mol/L and equal to or less than 2 mol/L.

For the electrolyte, a solid state electrolyte can also be used. In this case, a gel electrolyte or a perfect solid electrolyte can be used as the solid state electrolyte.

As a gel electrolyte, it is possible to use those prepared by adding an electrolyte or a salt melted at ambient temperature to a gelation agent. The gelation method can be implemented by techniques such as adding a polymer or an oil gelation agent, polymerizing coexisting polyfunctional monomers, or a crosslinking reaction of polymers. Examples of polymers used when gelation is performed by adding a polymer include polyacrylonitrile, polyvinylidene fluoride, and the like. Examples of oil gelation agents include dibenzylidene-D-sorbitol, cholesterol derivatives, amino acid derivatives, alkylamide derivatives of trans-(1R,2R)-1,2-cyclohexanediamine, alkyl urea derivatives, N-octyl-D-gluconamide benzoate, double-headed amino acid derivatives, quaternary ammonium salt derivatives, and the like.

When polyfunctional monomers are used for polymerization, the monomer to be used is preferably a compound having two or more ethylenic unsaturated groups. Examples of such monomers include divinylbenzene, ethylene glycol dimethacrylate, ethylene glycol diacrylate, diethylene glycol dimethacrylate, diethylene glycol diacrylate, triethylene glycol dimethacrylate, triethylene glycol diacrylate, pentaerythritol triacrylate, trimethylolpropane triacrylate, and the like.

Monofunctional monomers may be added in addition to the above polyfunctional monomers. Examples of the monofunctional monomers include esters or amides derived from acrylic acid or α-alkyl acrylates, such as acrylamide, N-isopropyl acrylamide, methyl acrylate, and hydroxyethyl acrylate; esters derived from maleic acid or fumaric acid, such as dimethyl maleate, diethyl fumarate, and dibutyl maleate; dienes such as butadiene, isoprene, and cyclopentadiene; aromatic vinyl compounds such as styrene, p-chlorostyrene, and sodium styrene sulfonate; vinyl esters such as vinyl acetate; nitriles such as acrylonitrile and methacrylonitrile; vinyl compounds having a nitrogen-containing heterocycle, such as vinyl carbazole; vinyl compounds having a quaternary ammonium salt; N-vinylformamide; vinyl sulfonate; vinylidene fluoride; vinyl alkyl ethers; N-phenyl maleimide; and the like. The proportion of the polyfunctional monomer to the total amount of the monomers is preferably equal to or more than 0.5% by mass and equal to or less than 70% by mass, and more preferably equal to or more than 1.0% by mass and equal to or less than 50% by mass.

The above monomers can be polymerized by radical polymerization. The radical polymerization of the monomers for the gel electrolyte can be performed by heating, light, ultraviolet rays, an electron beam or by an electrochemical method. Examples of polymerization initiators used for forming crosslinked polymers by heating include azo-based initiators such as 2,2′-azobis(isobutyronitrile) and 2,2′-azobis(dimethylvaleronitrile), peroxide-based initiators such as benzoyl peroxide, and the like. The amount of the polymerization initiator added is preferably equal to or more than 0.01% by mass and equal to or less than 15% by mass, and more preferably equal to or more than 0.05% by mass and equal to or less than 10% by mass, based on the total amount of the monomers.

When the electrolyte is gelated by the crosslinking reaction of the polymer, it is desirable to concurrently use a polymer and a crosslinking agent containing reactive groups necessary for the crosslinking reaction. Preferable crosslinkable reactive groups include nitrogen-containing heterocycles such as a pyridine ring, an imidazole ring, a triazole ring, an oxazole ring, a triazole ring, a morpholine ring, a piperidine ring, and a piperazine ring. Examples of preferable crosslinking agents include bi- or higher functional reagents that can cause an electrophilic substitution reaction with respect to nitrogen atoms, such as an alkyl halide, an aralkyl halide, a sulfonic acid ester, an acid anhydride, an acid chloride, and an isocyanate.

As the perfect solid electrolyte, a mixture of an electrolyte and an ion-conducting polymer compound can be used. Examples of the ion-conducting polymer compound include polar polymer compounds such as polyethers, polyesters, polyamides, and polysulfides.

When an inorganic solid electrolyte is used as the electrolyte, copper iodide, copper thiocyanide, or the like can be introduced to the inside of the electrode by a method such as casting, coating, spin coating, dipping, or electrolytic plating.

In the present embodiment, an organic hole-transporting material can be used instead of an electrolyte. Examples of the organic hole-transporting material include 2,2′,7,7′-tetrakis(N,N-di-p-methoxyphenylamine)-9,9′-spirobifluorene (Adv. Mater. 2005, 17, 813), aromatic diamines such as N,N′-diphenyl-N,N′-bis(3-methylphenyl)-(1,1′-biphenyl)-4,4′-diamine (U.S. Pat. No. 4,764,625), triphenylamine derivatives (JP-A-4-129271), stilbene derivatives (JP-A-2-51162), hydrazone derivatives (JP-A-2-226160), and the like.

The organic hole-transporting material can be introduced to the inside of the electrode by a method such as vacuum vapor-deposition, casting, spin coating, dipping, or electrolytic polymerization.

The method of preparing the electrolyte layer 5 of the present embodiment is roughly classified into two methods. One of the methods is a method of sticking in advance the counter electrode 8 onto the semiconductor layer 1 caused to adsorb a dye and interposing the liquid state electrolyte layer 5 in a gap therebetween, and the other method is a method of directly forming the electrolyte layer 5 on the semiconductor layer 1. In a case of the latter, the counter electrode 8 is formed on the electrolyte layer 5 after the electrolyte layer 5 is formed.

### EXAMPLES

Hereinbelow, the present invention will be described in more detail based on examples.

### Example 1

### Synthesis of Pyrroline-Based Compound P1

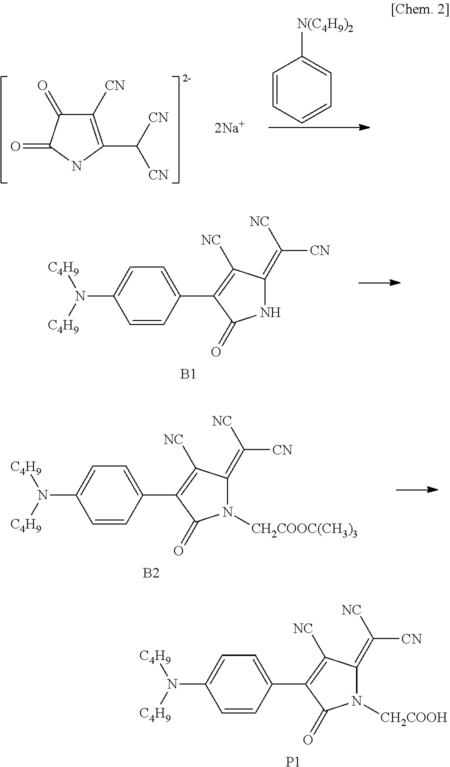

A 4-cyano-5-dicyanomethylene-3-hydroxy-2-oxo-3-pyrroline-disodium salt (U.S. Pat. No. 3,013,013) (7.5 g) and 8.25 g of N,N-dibutylaniline (manufactured by Wako Pure Chemical Industries, Ltd., product code 048-07803) were dissolved in 75 ml of N,N-dimethylformamide (manufactured by Wako Pure Chemical Industries, Ltd., product code 045-02916). Phosphorous oxychloride (manufactured by Wako Pure Chemical Industries, Ltd., product code 165-02282) was added dropwise thereto in a quantity of 15 g under ice cooling, followed by stirring for an hour under ice cooling and for another 4 hours at ambient temperature. The reaction mixture was poured into 1000 ml of ice water, and the precipitated crystals were filtered and washed several times with hot water. Subsequently, the resultant was stirred and washed three times under heating in 200 ml of ethanol (for industrial use, manufactured by KANTO KAGAKU)/acetonitrile (manufactured by Wako Pure Chemical Industries, Ltd., product code 014-00386) (=2/3), thereby obtaining 7.06 g of a compound B1 (yield 60%).

Thereafter, 6 g of the compound B1 and 4.9 g of t-butyl bromoacetate (manufactured by Wako Pure Chemical Industries, Ltd., product code 028-08962) were dissolved in 200 ml of acetonitrile, and 2.29 g of potassium carbonate (Wako Pure Chemical Industries, Ltd., product code 162-03495) was added thereto, followed by stirring at 80° C. for 3.5 hours. The resultant was left to cool and then poured into 1.5 L of ice water, and the precipitated crystals were filtered. The crystals were stirred and washed under heating in 150 ml of ethanol, followed by filtering, thereby obtaining 5.5 g of a compound B2 (yield 68%).

Subsequently, 4.5 g of B2 and 2.1 g of p-toluenesulfonic acid-hydrate (manufactured by Wako Pure Chemical Industries, Ltd., product code 207-03402) were dissolved in 100 ml of acetonitrile, followed by refluxing under heating for 6 hours. The resultant was left to cool and then poured into 1000 ml of ice water, and the precipitated crystals were filtered and washed with water. The crystals were then dissolved in acetone (for industrial use, manufactured by KANTO KAGAKU) and reprecipitated and purified in hexane (manufactured by Wako Pure Chemical Industries, Ltd., product code 085-00416)/ethyl acetate (manufactured by Wako Pure Chemical Industries, Ltd., product code 051-00356) (=10/1), thereby obtaining 3 g of a target pyrroline-based P1 (yield 75%).

Results of ¹H-NMR (acetone-d6) measurement of the obtained compound P1 were as follows. That is, δ was 11.2-12.7 (1H, br), 8.50 (2H, d), 7.03 (1H, d), 4.94 (2H, s), 3.63 (4H, t), 1.66-1.73 (m, 4H), 1.41-1.47 (m, 4H), 0.97 (6H, t).

λmax of the obtained dye in acetonitrile was 646 nm.

### Example 2

### Synthesis of Pyrroline-Based Compound P2

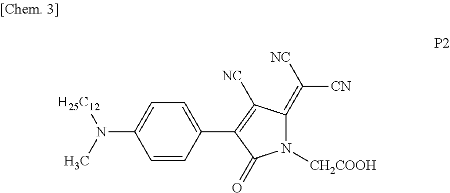

A pyrroline-based compound P2 was synthesized in the same manner as in Example 1. Here, N,N-dodecyl-N-methylaniline (synthesized by the method disclosed in Bull. Chem. Soc. Jpn., 68, pp 929-934 (1995)) was used instead of N,N-dibutylaniline.

Results of ¹H-NMR (acetone-d₆) measurement of the obtained compound were as follows. That is, δ was 11.2-12.7 (1H, br), 8.49 (2H, d), 7.02 (1H, d), 4.93 (2H, s), 3.66 (4H, t), 3.28 (s, 3H), 1.7-1.75 (m, 2H), 1.2-1.45 (m, 18H), 0.86 (3H, t).

λmax of the obtained dye in acetonitrile was 638 nm.

### Example 3

### Synthesis of Pyrroline-Based Compound P3

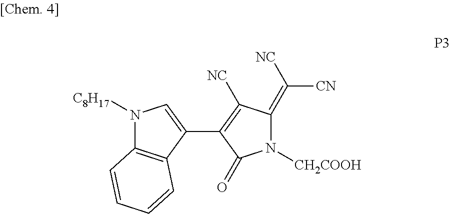

A pyrroline-based compound P3 was synthesized in the same manner as in Example 1. Here, N-octylindole (synthesized based on the method disclosed in J. Chem. Research(S), PP 88-89, 1984) was used instead of N,N-dibutylaniline.

Results of ¹H-NMR (acetone-d₆) measurement of the obtained compound were as follows. That is, δ was 11.2-12.7 (br, 1H), 8.76 (s, 1H), 8.30 (d, 1H), 7.75 (d, 1H), 7.39-7.47 (m, 2H), 5.00 (s, 2H), 4.51 (t, 2H), 1.97 (t, 2H), 1.2-1.47 (m, 18H), 0.84 (t, 3H).

λmax of the obtained dye in acetonitrile was 549 nm.

### Example 4

### Synthesis of Pyrroline-Based Compound P4

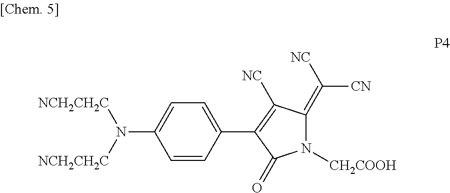

A pyrroline-based compound P4 was synthesized in the same manner as in Example 1. Here, N,N-bis(2-cyanoethyl)aniline (manufactured by Wako Pure Chemical Industries, Ltd., product code 327-30172) was used instead of N,N-dibutylaniline.

Results of ¹H-NMR (acetone-d₆) measurement of the obtained compound were as follows. That is, δ was 11.2-12.7 (br, 1H), 8.46 (d, 2H), 7.25 (d, 2H), 4.97 (s, 2H), 4.13 (t, 4H), 2.97 (t, 4H).

λmax of the obtained dye in acetonitrile was 579 nm.

### Example 5

### Preparation of Photoelectric Conversion Element

<<Preparation of Semiconductor Electrode and Counter Electrode>>

First, a semiconductor electrode was prepared in the following sequence.

FTO-attached glass (10 Ωcm²) (15 mm×15 mm) having a thickness of 1.1 mm was prepared as a conductive substrate (transparent conductive layer-attached light transmissive substrate).

Subsequently, 5 g of commercially available titanium oxide powder (product name: P25, manufactured by Nippon Aerosil Co., Ltd.), 20 ml of a 15 vol % aqueous acetic acid solution, 0.1 ml of a surfactant (product name: Triton (registered trademark) X-100, manufactured by Sigma-Aldrich Co. LLC.), and 0.3 g of polyethylene glycol (molecular weight 20000) (manufactured by Wako Pure Chemical Industries, Ltd., product code 168-11285) were stirred for about an hour in a stirring mixer, thereby preparing a titanium oxide paste as a material of a semiconductor layer.

Thereafter, the titanium oxide paste was coated (coating area: 10 mm×10 mm) in an appropriate amount onto the FTO-attached glass by a doctor blade method so as to yield a film thickness of about 50 μm. The FTO-attached glass coated with the titanium oxide paste was then inserted into an electric furnace and baked at 450° C. for about 30 minutes in the atmosphere, followed by natural cooling, thereby forming a porous titanium oxide semiconductor layer as a semiconductor layer.

Subsequently, in order to form a light scattering layer, a paste was prepared by mixing the above titanium oxide paste with titanium oxide having an average particle size of 300 nm such that the ratio of the titanium oxide to the titanium oxide paste became 20% by weight. This paste was coated by a screen printing method onto the above porous titanium oxide semiconductor layer to yield a thickness of 20 μm. The resultant was baked at 450° C. for about 30 minutes in the atmosphere and cooled naturally.

In addition, a platinum layer having an average film thickness of 1 μm as a catalytic layer was vapor-deposited by a vacuum vapor deposition method onto a soda lime glass plate (thickness of 1.1 mm), thereby preparing a counter electrode.

<<Dye Adsorption>>

Thereafter, a dye was adsorbed onto the surface of the semiconductor layer formed of the above thin titanium oxide film. For the dye, a solution was used which was prepared by dissolving the pyrroline-based compound P3 synthesized in Example 3 in acetonitrile at a concentration of about 2×10⁻⁴ M. The semiconductor electrode having the above porous titanium oxide semiconductor layer was dipped in this dye solution and stored overnight. Subsequently, the semiconductor electrode was taken out of the dye solution, rinsed with acetonitrile to remove the surplus dye, and then dried in the air.

<<Cell Assembly>>

The semiconductor electrode having undergone the dye adsorption treatment and the above counter electrode were arranged such that the semiconductor layer and the catalytic layer face each other. Thereafter, the periphery of the cell portion was thermally compressed using a thermosetting resin film in which cuts were made such that the electrolyte layer could permeate the gap.

<<Injection of Electrolyte Layer>>

An iodine-based electrolyte as the electrolyte layer was injected into the above cell from the counter electrode side by using surface tension. The iodine-based electrolyte was prepared by adjusting the concentration by using methoxypropionitrile (manufactured by Wako Pure Chemical Industries, Ltd., product code 134-12225) for a solvent such that iodine (manufactured by Wako Pure Chemical Industries, Ltd., product code 092-05422) had a concentration of 0.5 mol/L, lithium iodide (manufactured by Wako Pure Chemical Industries, Ltd., product code 122-03452) had a concentration of 0.1 mol/L, 4-tert-butyl pyridine (manufactured by Tokyo Chemical Industry Co., Ltd., product code B0388) had a concentration of 0.5 mol/L, and 1,2-dimethyl-3-propyl imidazolium iodide (manufactured by Tokyo Chemical Industry Co., Ltd., product code D3903) had a concentration of 0.6 mol/L.

<<Photocurrent Measurement>>

Light having an intensity of 100 mW/cm² under a condition of AM 1.5 was emitted to the photoelectric conversion element prepared in the above manner by using a solar simulator. The generated electricity was measured with a current and voltage measurement instrument, and the photoelectric conversion characteristics were evaluated. As a result, a photoelectric conversion efficiency of 4.3% could be obtained.

### Example 6

A photoelectric conversion element was prepared in the same manner as in Example 5. Here, the pyrroline-based dye P4 was used instead of the pyrroline-based dye P3. The photoelectric conversion characteristics of the obtained element were evaluated, and as a result, a photoelectric conversion efficiency of 3.8% could be obtained.

As clearly shown in the above description, it is possible to obtain excellent photoelectric conversion efficiency by using the pyrroline-based compound of the present invention for a dye for photoelectric conversion. This photoelectric conversion element of the present invention is usable for a semiconductor electrode, a photoelectric conversion element, a solar cell, and the like.

The present application claims priority based on Japanese Patent Application No. 2010-002241 filed Jan. 7, 2010, the entire disclosure of which is employed herein.

1. A dye for photoelectric conversion comprising:
at least one or more kind of a compound represented by the following General Formula (1), 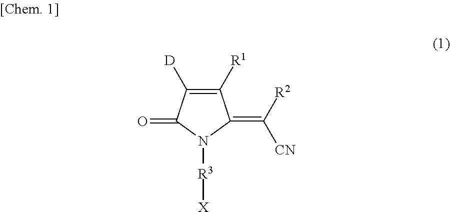
(in Formula (1), R¹ and R² represent any one of —CN, —SO₂R, —COOR, and —CONR₂ (R represents a hydrogen atom, a substituted or unsubstituted alkyl group, a cycloalkyl group, or an aryl group); R³ represents a direct bond or a substituted or unsubstituted alkylene group; X represents an acidic group; and D represents an organic group having an electron donating substituent or a substituted or unsubstituted heterocyclic group).

2. The dye for photoelectric conversion according to claim 1,
wherein the acidic group is a carboxy group, a hydroxy group, a sulfonic acid group, or a phosphonic acid group.

3. The dye for photoelectric conversion according to claim 1,
wherein the organic group having an electron donating substituent is an aryl group having an electron donating substituent.

4. A semiconductor electrode comprising:
a semiconductor layer onto which at least one or more kind of the dye for photoelectric conversion according to claim 1 has been adsorbed.

5. The semiconductor electrode according to claim 4,
wherein the semiconductor layer is constituted with a semiconductor material containing titanium oxide or zinc oxide.

6. A photoelectric conversion element using the semiconductor electrode according to claim 4.

7. A solar cell comprising:
the photoelectric conversion element according to claim 6.

8. A compound represented by the following General Formula (1), 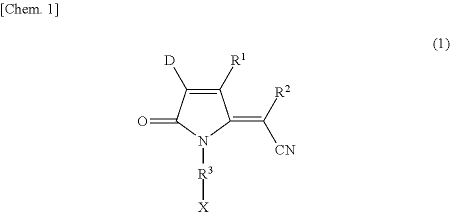
(in Formula (1), R¹ and R² represent any one of —CN, —SO₂R, —COOR, and —CONR₂ (R represents a hydrogen atom, a substituted or unsubstituted alkyl group, a cycloalkyl group, or an aryl group); R³ represents a direct bond or a substituted or unsubstituted alkylene group; X represents an acidic group; and D represents an aryl group having an electron donating substituent or a substituted or unsubstituted heterocyclic group).
